(19) United States
(12) Patent Application Publication
(10) Pub. No.: US 2009/0270915 A1
(43) Pub. Date: Oct. 29, 2009

(54) Portable chiropractic adjustor

(76) Inventors: Stephen C. Tsai, Fullerton, CA (US); Arlan W. Fuhr, Phoenix, AZ (US)

Correspondence Address: GREENBERG TRAURIG (PHX), INTELLECTUAL PROPERTY DEPARTMENT, 2450 COLORADO AVENUE , SUITE 400E, SANTA MONICA, CA, 90404, US

(21) Appl. No.: 12/220,372

(22) Filed: Jul. 24, 2008

Related U.S. Application Data
(60) Provisional application No. 60/951,704, filed on Jul. 24, 2007.

(51) Int. Cl. A61F 5/00

(52) U.S. Cl. 606/238

(57) ABSTRACT

A portable battery power operated chiropractic adjustor, manipulator or thruster for applying an adjustment energy to a patient through a plunger having a resilient or cushioned head with the energy applied to the plunger being supplied by non-manual sources and the impulse is adjustable or tunable along with having annunciators or indicators for preload and readiness to operate. The power source may be an internal rechargeable battery or removable rechargeable battery pack and the adjustor is DC motor operated to impart selectively single or multiple thrusts.

### FIELD OF INVENTION

The present invention relates generally to a portable chiropractic adjustor for use in chiropractic adjustment of musculoskeletal structures. More, particularly, this invention concerns an improved power operated chiropractic-adjusting device for use in spinal manipulative therapy to apply impact forces or thrusts to a human body.

### BACKGROUND

The chiropractic art is generally concerned with adjusting misaligned body structures by manually manipulating the various joints in the human body. Of more specific interest in the art, however, is the spinal column which is comprised of several interconnected musculoskeletal structures or vertebrae. Unlike other, less critical body structures, the spinal column must be treated or manipulated with extreme caution because of its link with the central nervous system.

The human spine is susceptible to many different pathologic abnormalities including misalignment, miscellaneous trauma and pain, and degeneration as a result of age or disease. By employing various physical therapy techniques, though, a chiropractor, or one skilled in the chiropractic art, may be able to successfully treat a pathologic spine. Successful treatment will not only relieve any pain or discomfort that the patient might be suffering, but will also improve the overall quality of life of that patient.

One common spinal-adjustment technique involves applying thrusts or forces to the afflicted region of the spine. In particular, this technique involves either “mobilizing” the spine (i.e. passively moving the spine with relatively slow cyclic or oscillatory motion), or “manipulating” the spine (i.e. applying an impulsive thrust or force in a well-defined direction to a specific region of the spine). Depending on professional affiliations, this technique is referred to as chiropractic adjustment, osteopathic manipulation, orthopedic manual therapy, and/or spinal manipulative therapy.

There are several well-known procedures or techniques for “manipulating” or administering impulsive thrusts to a spine. One technique involves applying one or more rapid thumb thrusts to misaligned or afflicted vertebrae. Thumb thrusts, however, tend to be both imprecise in magnitude and location and tiresome to administer. Another technique involves using a manually operated chiropractic-adjusting instrument. For instance, U.S. Pat. No. 4,116,235, issued to Fuhr et al. (“Fuhr”); Fuhr U.S. Pat. No. 6,702,836; Fuhr U.S. Pat. No. 6,379,375; Keller et al. U.S. Pat. No. 5,626,615; Keller et al. U.S. Pat. No. 5,656,017; and U.S. Pat. No. 4,498,464, issued to Morgan, Jr., disclose such instruments.

Throughout the years it has also been known that power driven devices at times can offer benefits or advantages in use over the manually operated devices. Particularly, there is a current need for a compact, lightweight device that is portable and yet can be easily and repetitively operated by a person with a lower degree of hand strength.

Electric solenoid operated adjustors such as ones described in Evans U.S. Pat. No. 4,841,955 issued in 1989 or Adelman U.S. Pat. No. 4,682,490, issued in 1987, can provide adjusting and controllability benefits over manual devices. However, using an electrical appliance operating at conventional voltages close to the body can be potentially hazardous and even prohibited by governmental regulatory agency rules or regulations and power supply cords can get in the way.

Thus, numerous efforts have been made to develop a power operated thrusters with all of the desired features and benefits required for safe and varied usage of such devices. Examples of such an approach in pneumatic operated thrusters is shown in U.S. Pat. No. 4,716,890, issued in 1988 to Bichel.

While the Bichel thruster as described did seek to overcome disadvantages presented in prior art devices, it still did not provide certain features and advantages required to achieve wide spread acceptance and use by chiropractic practitioners.

By way of example, it may be noted that such prior devices including Bichel are capable of only delivering a single thrust or stroke, provide only manual adjustability of stroke lengths; provide force adjustment by changing stroke length and change air pressure only at the compressor or supply source. In addition they involve complicated multiple parts designs which make them more costly to manufacture and more difficult and costly to maintain or use. A pneumatic thruster of Frye U.S. Pat. No. 6,503,211 has solved some of the problems and objections of the field.

### SUMMARY OF THE INVENTION

The present invention may provide a portable chiropractic adjustor instrument comprising a DC motor driven plunger. The instrument may be relatively light weight and have a substantially long life rechargeable battery.

The present invention may provide an improved power operated chiropractic instrument that is “tunable” or settable as to load, amplitude, and frequency within a user selected range of natural frequency.

The present invention may provide a portable device with annunciators or indicators of settings such as preload and readiness to operate.

The present invention may provide a self contained power source for the adjusting instrument which is long lasting and yet can be rechargeable or replaceable.

These together with other features of the invention are more fully hereinafter described, reference being had to the accompanying drawings forming a part hereof, wherein like numerals refer to like parts throughout.

It will be appreciated that the foregoing and subsequent descriptions are considered as illustrative only of aspects and features of the invention. Thus, it is not desired to limit the invention to the exact construction and operation shown and described, and, accordingly, all suitable modifications and equivalents may be utilized, falling within the scope of the invention.

### BRIEF DESCRIPTION OF THE DRAWINGS

FIG. 1 is a top plan view of an embodiment of a portable chiropractic adjustor of the present invention;

FIG. 2 is a right elevational side view of the portable chiropractic adjuster shown in FIG. 1;

FIG. 3 is a left elevational side view of the portable chiropractic adjuster shown in FIG. 1;

FIG. 4 is a bottom view of the portable chiropractic adjuster shown in FIG. 1;

FIG. 5 is a front elevational view of the portable chiropractic adjuster shown in FIG. 1;

FIG. 6 is a rear elevational view of the portable chiropractic adjuster shown in FIG. 1;

FIG. 7 is a partial cross-sectional view taken through line 7-7 of the portable chiropractic adjuster shown in FIG. 1;

FIG. 8 is another cross-sectional view taken through line 7-7 of the portable chiropractic adjuster shown in FIG. 1;

FIG. 9 is a cross-sectional view taken through line 9-9 of FIG. 2 of the portable chiropractic adjuster shown in FIG. 1;

FIG. 10 is an exploded view of the portable chiropractic adjuster shown in FIG. 1;

FIG. 11 is an exploded view of a motor and gear mechanism for the portable chiropractic adjuster shown in FIG. 1;

FIG. 12 is an exploded view of a heat dissipating mechanism for the portable chiropractic adjuster shown in FIG. 1;

FIG. 13 is an exploded view of a thrust transfer mechanism for the portable chiropractic adjuster shown in FIG. 1;

FIG. 14 is an exploded view of a barrel portion for the portable chiropractic adjuster shown in FIG. 1;

FIG. 15 is an exploded view of a plunger mechanism for the portable chiropractic adjuster shown in FIG. 1; and

FIG. 16 is a simplified diagram illustrating a control board in communication with components of the portable chiropractic adjuster shown in FIG. 1.

### DETAILED DESCRIPTION OF THE INVENTION

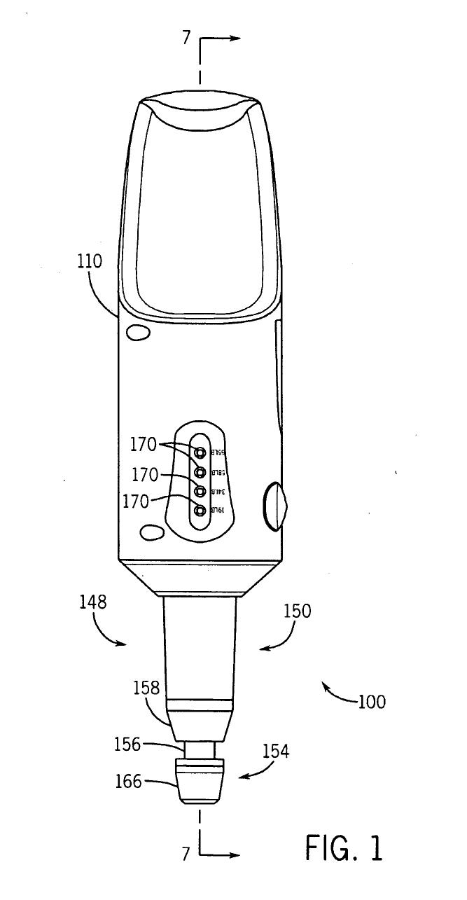

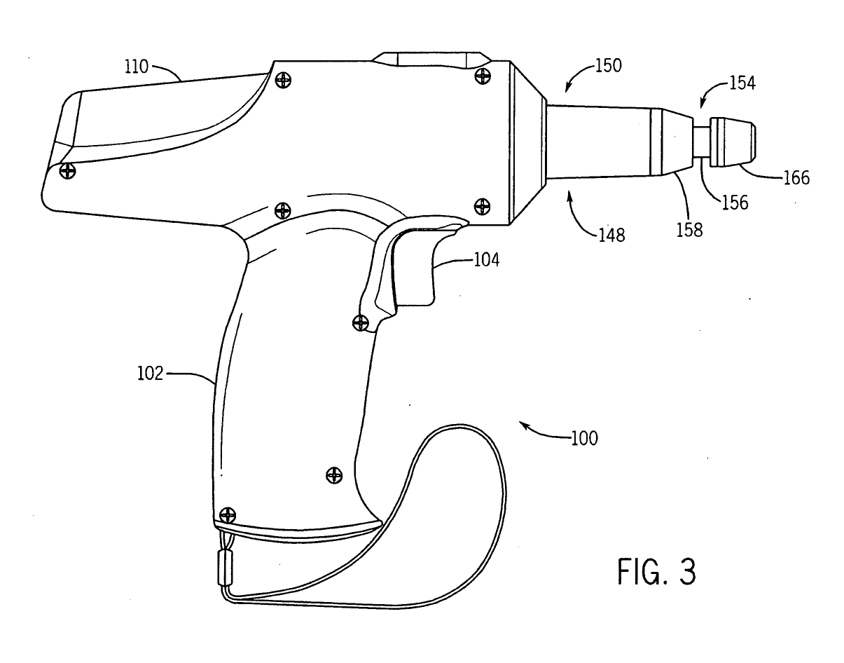

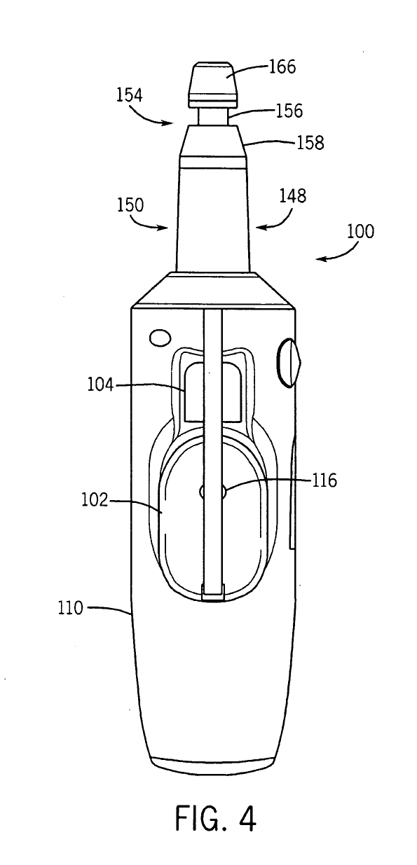

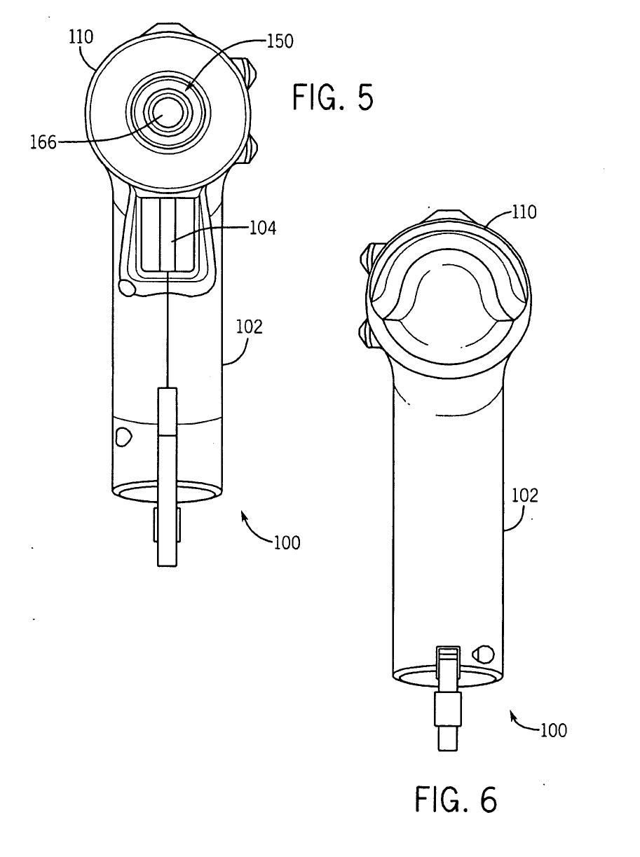

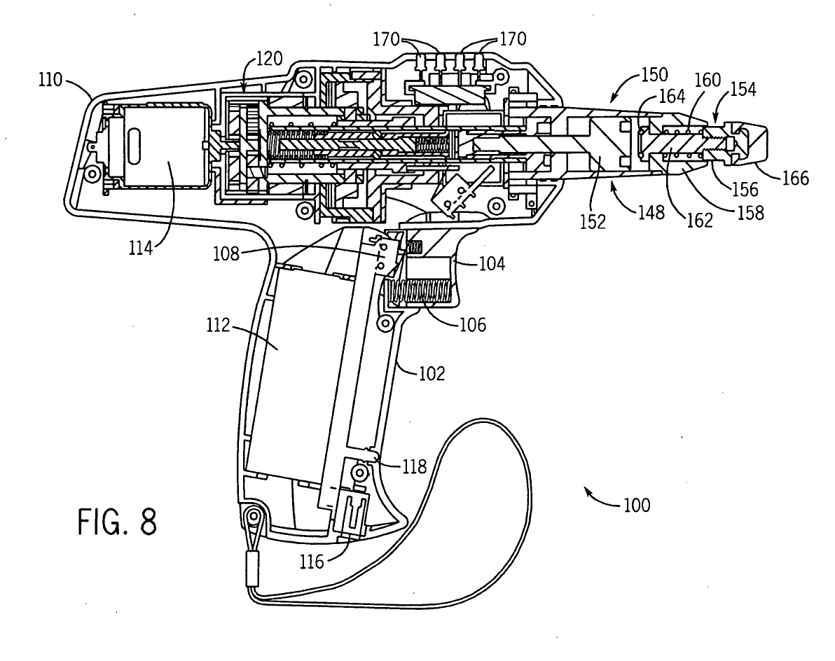

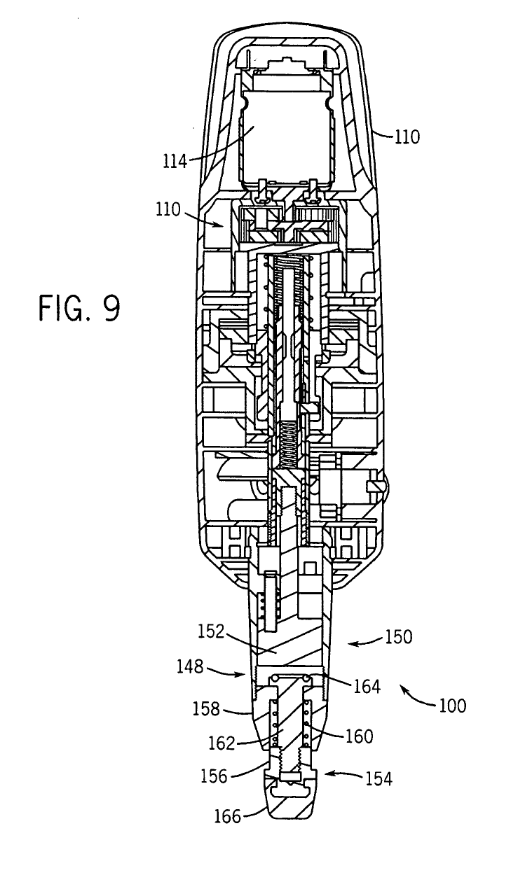

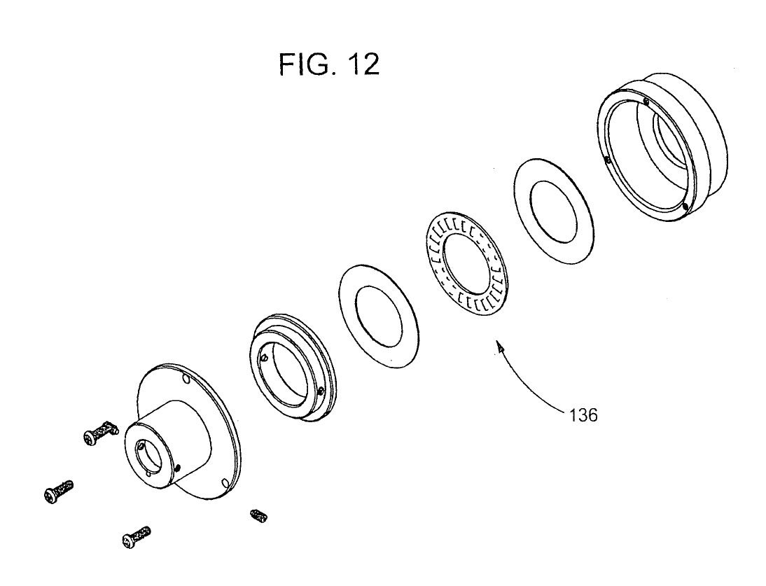

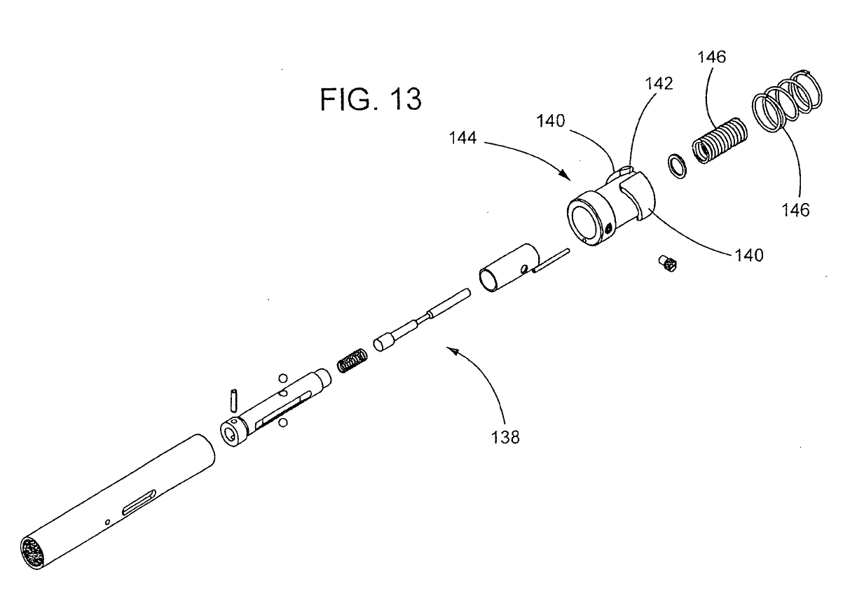

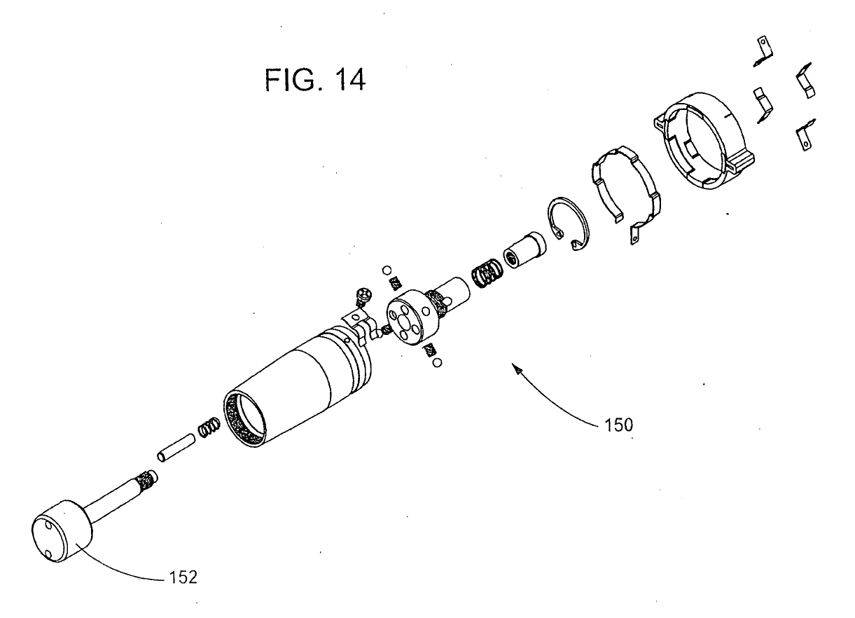

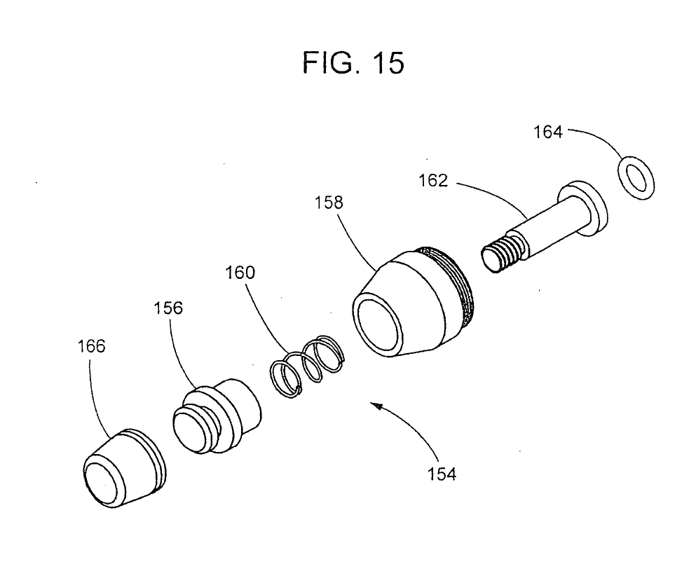

Turning to FIGS. 1-16, there is shown another embodiment of a portable chiropractic adjustor instrument 100. Referring to FIGS. 1-6, the portable chiropractic instrument 100 may comprise a structure, including a housing, that is relatively convenient for an operator to hold, adjust, and operate. The instrument may have an ergonomic handle 102 which is comfortable and convenient to grip. The portable chiropractic instrument 100 may also have a trigger mechanism 104 that likewise comfortably receives an operators finger for firing the instrument. As shown in FIG. 8, the trigger 104 may be coupled to a spring 106 which biases the trigger 104 in the non-engaged position. The trigger 104 may be in contact with a switch 108 that is depressed when the trigger 104 is depressed to operate the instrument 100.

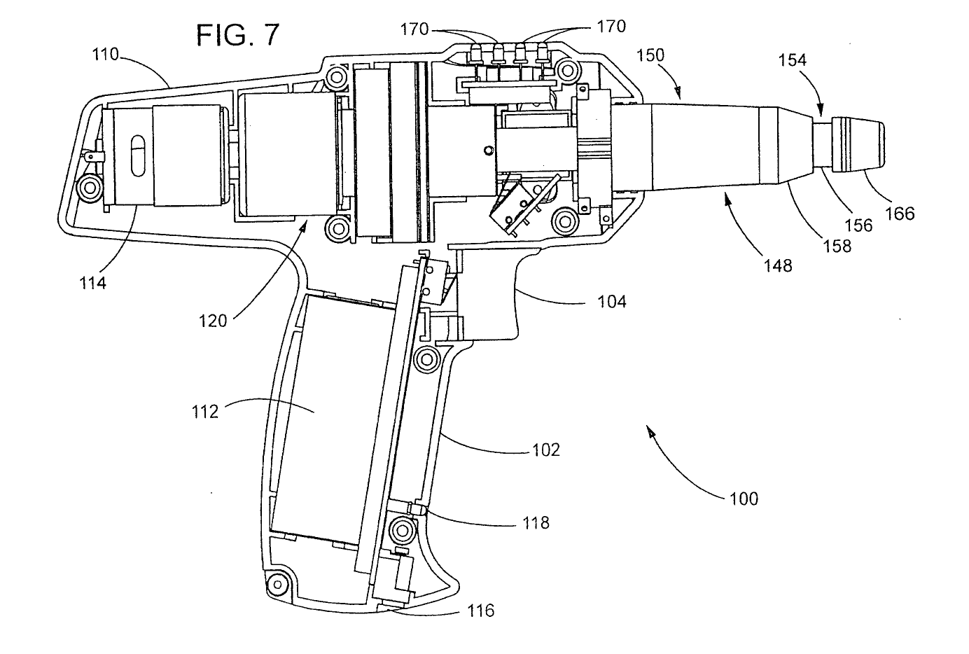

Turning to FIGS. 7-15, suitable internal components of the portable chiropractic instrument 100 are shown in detail. The instrument 100 may utilize a battery 112 such that the such that the portability of the instrument 100 is enhanced by not requiring an unwieldy power cord for usage. Furthermore, in order to offer further enhanced portability along with a reasonable battery usage life, the instrument may utilize a DC motor 114 which is significantly more energy efficient than solenoid driven devices. In addition, a DC motor 114 may generally be constructed such that it weighs less than a solenoid for supplying a comparable amount of force such that the instrument 100 may be made lighter, and thus, easier for an operator to hold over an extended period of time than a solenoid driven instrument. The battery 112 may be disposed in the handle 102 and the motor 114 may be disposed in the housing 110 such that it is in line with a plunger mechanism 154 (described in further detail below).

In some embodiments, the battery 112 may be generally disposed within the housing 110 such that it is not generally removable by the operator. In other embodiments, the battery 112 may be removable and replaceable by the operator. The battery 112 may be any suitable battery for driving a DC motor 114 with long usage and recharge characteristics. The battery 112 may be rechargeable such that the battery 112 may be reused after charging.

The instrument 100 may comprise a charging port 116, such as shown in FIGS. 7 and 8, for receiving an adaptor to recharge the battery 112. In some embodiments where the battery 112 is removable, the battery 112 may be removed for charging in a free standing charger and may be replaced with a spare charged battery when one battery 112 is being charged. The instrument 100 may also comprise a charge indicator 118, such as an LED light shown in FIGS. 7, 8, and 10, that may inform an operator when the battery 112 is charging, when the battery 112 has been charged, and/or when the battery 112 is nearly discharged. It will be appreciated that any suitable form of battery 112 indicator may be used, the instrument 100 may utilize any suitable number of battery indicators, and the battery indicator(s) may be used to indicate any suitable battery condition. In some embodiments, the battery 112 may be a lithium-ion rechargeable battery.

Referring to FIGS. 7-11, the motor 114 may be in contact with a gear mechanism 120 that may comprise a plurality of gears 122 for assisting with the transfer of force from the motor 114 to further components of the instrument 100. In some embodiments, the gear mechanism 120 may comprise a gear housing 124 having gear teeth 126 on an internal surface of the gear housing 124. The gear mechanism 120 may comprise one or more sets of planetary gears 128. A pinion gear plate 130 may be disposed between sets of planetary gears 128 to transfer motion. The gear mechanism 120 may also comprise a head portion 132 having one or more camming actuators, such as protrusions 134.

Referring to FIGS. 7-10 and 12, the instrument may comprise a heat dissipation structure 136 for dissipating heat within the instrument 100. In some embodiments, the heat dissipation structure 136 may operate as a heat sink to draw heat away from one or more components within the instrument 100. The heat dissipation structure 136 may at least partially surround a portion of the thrust transfer mechanism 138 (described below).

Turning to FIGS. 7-10 and 13, the instrument 100 may comprise a thrust transfer mechanism 138. The thrust transfer mechanism 138 may comprise a suitable structure(s) for contacting the gear mechanism and converting the rotational forces conveyed by the motor 114 to the gear mechanism 120 into an axial force directed away from the motor 114 and toward a patient. For example, the thrust transfer mechanism 138 may operate as a camming structure and may comprise one or more ramping structures 140, with grooves 142 therebetween, coupled to a ramping component 144. The protrusions 134 of the head portion 132 of the gear mechanism 120 may ride along these ramping structures 140. The ramping component 144 may be biased away from the motor by one or more springs 146.

As the head portion 132 of the gear mechanism 120 is rotated, the protrusions 134 ride up the ramping structures 140 which forces the ramping component 144 to move toward the motor 114 and compress the one or more springs 146. As the ends of the ramping structures 140 are reached, the protrusions 134 reach the grooves 142 and the spring force propels the ramping component 144 forward. In this way, the rotational forces of the motor 114 have been converted into an axial force.

Referring to FIGS. 1-5, 7-10, and 14, the instrument 100 may comprise a forward end 148 having a barrel portion 150 coupled to the housing 110. The barrel portion 150 may comprise a plurality of components for receiving a force from the thrust transfer mechanism 138 and conveying the force to a plunger mechanism 154 (described below). For example, the barrel portion 150 may comprise a hammer 152 for receiving and carrying forward the force created by the spring force propelling the ramping component 144 forward when the trigger 104 has been engaged.

Referring to FIGS. 1-5 and 7-10, and 15, the forward end 148 may further have a plunger mechanism 154. The plunger mechanism 154 may have a suitable structure for asserting a force on a patient. Furthermore, the plunger mechanism 154 may have a suitable structure for allowing an operator to assert a preload force on a patient before firing the instrument 100.

The plunger mechanism 154 may comprise a plunger 156, a plunger housing 158, a spring 160, a force transfer pin 162, and an o-ring 164. When assembled, the spring 160 may be disposed around the force transfer pin 162 and both the spring 160 and the force transfer pin 162 may be at least partially disposed within the plunger housing 158. The plunger housing 158 may be coupled to the barrel portion 150. A tip 166 may be disposed at an end of the plunger 156 for contact with the patient. The tip 166 may be cushioned. The plunger 156 may be mounted such that the plunger 156 may be at least partially received by the plunger housing 158. The plunger 156 can be interchangeable with various single or dual style forms.

The plunger 156 may be movable with respect to the plunger housing 158 when a force is asserted against the tip 166. The plunger 156 may be coupled to a suitable structure, such as the spring 160 disposed against the force transfer pin 162, which applies an increasing amount of force to resist the inward movement of the plunger 156 as the plunger 156 is pushed further into the plunger housing 158. This permits an operator of the instrument 100 to apply a preload force prior to engaging the trigger 104. Therefore, the plunger mechanism 154 may have a structure such that as the instrument 100 is pressed against the patient and as the plunger 156 moves further into the plunger housing 158, the amount of preload force increases.

In addition, the force transfer pin 162 receives the force transmitted through the barrel portion 150 when the trigger 104 is engaged and directs the force onto the patient via the plunger 156 and plunger tip 166. This force is carried by the hammer 152 through the barrel portion 150 to the force transfer pin 162. The o-ring 164 provides a cushioning contact between the force transfer pin 162 and the hammer 152 to reduce the noise that would otherwise result from the impact of these two components and also reduce the wear on these components. The amount of preload force that is applied to the force transfer pin 162 may affect the distance that the force transfer pin 162 travels when the trigger 104 is engaged.

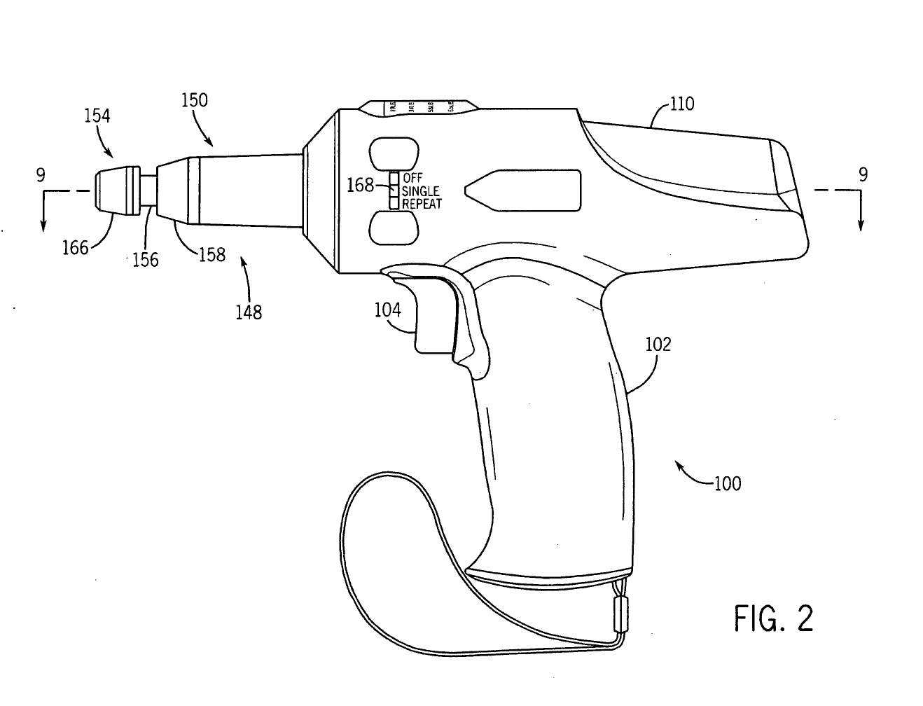

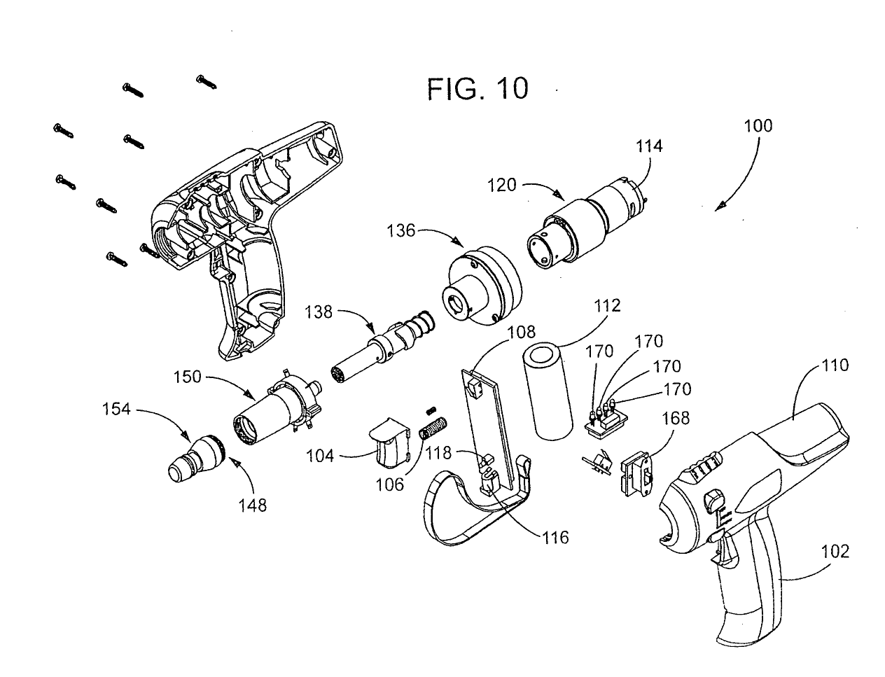

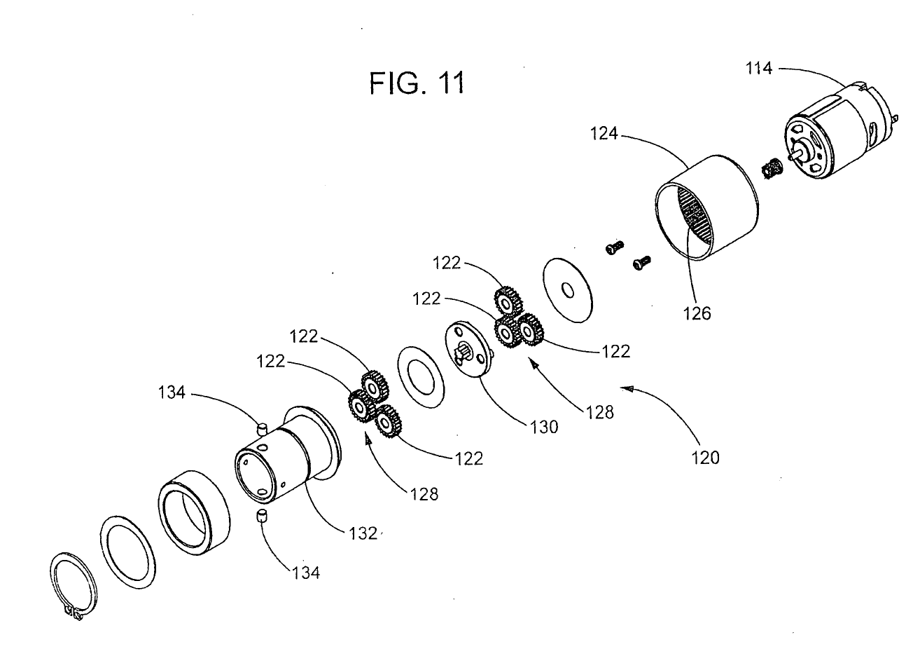

Referring to FIGS. 2 and 10, the instrument 100 may comprise a suitable mechanism for turning the instrument 100 on and off and setting the instrument 100 to any suitable operating mode. For example, the instrument 100 may comprise a switch 168 that is movable between any two or more suitable positions to place the instrument 100 into a desired operating mode.

In one embodiment, the switch 168 may be movable between an off mode, a single thrust operating mode, and a multiple thrust operating mode. In the single thrust operating mode, the instrument 100 may apply a single thrust for each activation of the trigger 104. In comparison, the multiple thrust setting may apply multiple thrusts for each activation of the trigger 104. In some embodiments, the instrument 100 may continue to apply thrusts until such time as the trigger 104 is released. In other embodiments, the instrument 100 may apply a predetermined or operator set number of thrusts. It will be appreciated, however, that the switch 168 may be used to place the instrument 100 into any suitable operating mode and the switch 168 may be movable between any suitable number of operating modes.

An annunciation structure may be disposed on the instrument to provide feedback to the operator as to such things as preload settings, loading status, readiness, force settings, and even values of impulse frequency or amplitude. It will be appreciated that the annunciation structure may be any suitable structure in any suitable quantity. As shown in FIGS. 1, 7, and 8, the housing may comprise a plurality of indicators 170, such as LED lights, that indicate the thrust setting. The instrument 100 may have any suitable number of indicators 170 representing any suitable number of thrust settings. In some embodiments, the LED lights could change colors, such as from red to yellow to green, for example, for preload status. In some embodiments, the annunciation structure may be an LCD display and/or an audio annunciator. An LCD display could be graphic, alpha numeric, and/or pictorial.

By way of example and not limitation, as shown in FIGS. 1, 7, and 8, the instrument may have indicators 170 that notify the operator when certain predetermined amounts of preload force have been asserted on the patient based on information provide by the one or more preload force sensors disposed within the housing 110. As the amount of preload force applied reaches a first predetermined amount, the first indicator will notify the operator. As the amount of preload force is increased to a second predetermined amount, the second indicator will notify the operator. Further indicators may be provided to indicate when further increased amounts of preload force have been reached. In this way, the operator may accurately apply a desired amount of preload force before engaging the trigger 104. In one example embodiment, the respective indicators 170 may notify an operator when 19 pounds, 34 pounds, 38 pounds, and/or 55 pounds of preload force have been applied. In another example embodiment, respective indicators 170 may notify an operator when 75 Newtons, 125 Newtons, 175 Newtons, and/or 250 Newtons of preload force have been applied. It will be appreciated that any suitable number of indicators 170 may indicate any suitable levels of preload force.

The motor 114 may be in contact with a gear mechanism 120 for driving the thrust of the tip 166 in response the engagement of the trigger 104. When the trigger switch 108 is depressed, power from the battery 112 may be provided to the motor 114. Depending on whether the instrument 100 is set for single thrust operation or multiple thrust operation, the motor 114 may be driven for only a relatively short period of time, the motor 114 may be driven for a desired longer period of time to provide a predetermined number of thrusts, or the motor 114 may be driven until the trigger 104 is released. The motor 114 may drive the gear mechanism 120, which in turn, may apply a force on the thrust transfer mechanism 138. The thrust transfer mechanism 138 may convert the rotational force of the gear mechanism 120 into an axial force that may be transmitted through the instrument to the plunger 156 and on the patient.

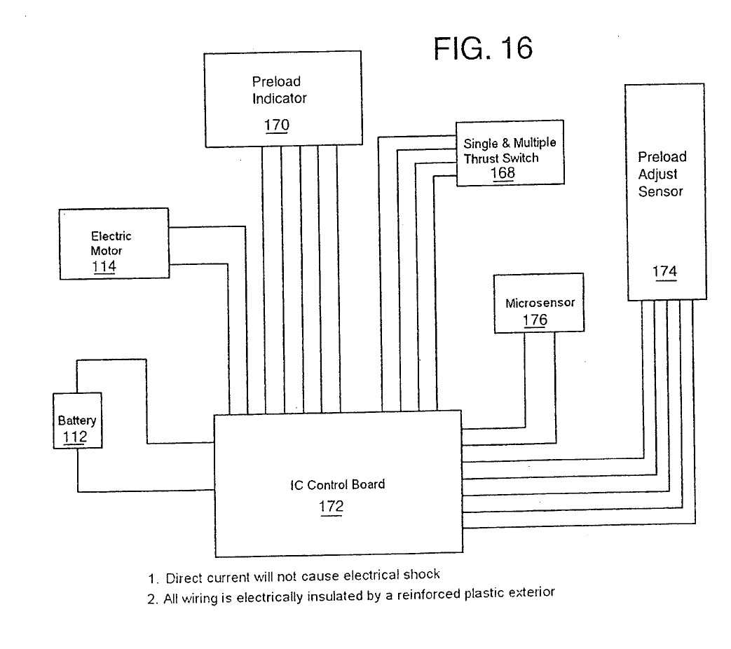

As shown in FIG. 16, a control board may be disposed within the instrument that may be used to communicate with such features as the battery 112, motor 114, annunciaton structure(s) (such as 170), thrust setting switch(es) 168, microsensors 176, preload sensor(s) 174, and any other suitable structures, for operation of the portable chiropractic instrument 100. The circuitry of the instrument 100 may also contain microprocessor devices for data storage such as number and duration of thrusts, for example, and the data collected can be downloadable to a computer having diagnostic software and/or a patient database.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods

what is claimed is:

1. A portable chiropractic adjustor for applying an adjustment energy to a patient, the portable chiropractic adjustor comprising:
a plunger mechanism having a plunger and a plunger tip;
a housing having a thrust transfer structure for transferring an axial thrust force through the housing to the plunger mechanism, a camming structure, and a handle having a trigger;
a DC motor disposed within the housing for rotatably driving the camming structure in response to the trigger being engaged; and
a cam actuator for converting the rotational drive of the camming structure to the axial thrust force applied to the thrust transfer structure.

2. The portable chiropractic adjustor of claim 1 wherein the DC motor is battery operated.

3. The portable chiropractic adjustor of claim 2 wherein the battery is rechargeable.

4. The portable chiropractic adjustor of claim 1 wherein the plunger mechanism has a spring in contact with the plunger for an application of a preload force.

5. The portable chiropractic adjustor of claim 1 further comprising an annunciator.

6. The portable chiropractic adjustor of claim 5 wherein the annunciator indicates an amount of preload force applied.

7. The portable chiropractic adjustor of claim 1 further comprising a switch for changing the portable chiropractic adjustor between a single thrust operation mode and a multiple thrust operation mode.

8. The portable chiropractic adjustor of claim 1 wherein the camming structure comprises a ramp for contacting the cam actuator.

9. The portable chiropractic adjustor of claim 8 wherein the camming structure comprises a groove for receiving the cam actuator.

10. The portable chiropractic adjustor of claim 1 wherein the camming structure is in contact with a spring.
